(19) United States
(12) Patent Application Publication
(10) Pub. No.: US 2013/0035565 A1
(43) Pub. Date: Feb. 7, 2013

(54) Method and System for the Reactivation of an Implantable Chemical Sensor

(71) Applicant: Jens Potschadtke, Erlangen (DE)

(72) Inventor: Jens Potschadtke, Erlangen (DE)

(73) Assignee: BIOTRONIK SE & CO. KG, Berlin (DE)

(21) Appl. No.: 13/553,119

(22) Filed: Jul. 19, 2012

Related U.S. Application Data
(60) Provisional application No. 61/514,490, filed on Aug. 3, 2011.

(51) Int. Cl. A61B 5/145

(52) U.S. Cl. 600/309

(57) ABSTRACT

A method for the operation and, in particular, for the reactivation of an implantable chemical sensor that measures values in bodily fluid, including the steps of wetting the sensor surface with a reactivation substance that carries out a chemical reaction on the sensor surface that removes molecules originating in the bodily fluid, or the reaction or degradation products thereof, which have collected on the sensor; and dewetting the sensor surface of the reactivation substance, wherein the sensor surface is wetted and dewetted with the reactivation substance in situ.

### CROSS-REFERENCE TO RELATED APPLICATIONS

This patent application claims the benefit of co-pending U.S. Provisional Patent Application No. 61/514,490, filed on Aug. 3, 2011, the disclosure of which is hereby incorporated by reference in its entirety.

### TECHNICAL FIELD

The present inventive disclosure relates to a method for the operation, and in particular for the reactivation, of an implantable sensor, and an implantable system comprising the sensor.

### BACKGROUND

Continuous real-time monitoring of clinical analytes, e.g., PO₂, PCO₂, K+, Na+, glucose and lactate, gas content, ions, proteins, antibodies, pH value, hormones, messenger substances, metabolites, pathogens, blood components, clotting factors, cholesterol, nutrients, toxins, drug components, and other measured values, plays an increasingly more decisive role in numerous therapies for preventive medical care. For this reason, the requirements on implantable chemical sensors that monitor the physiological state of a patient in real time or within the biological environment or both are increasing. The specific principle of measurement employed by a sensor depends greatly on the type of substances to be measured. A sensor can be, e.g., an electrochemical sensor that measures the pH value. Other known sensor principles are adsorption sensors that can detect the adsorption of substances (molecules or ions) on the basis of the change in the surface properties. The surface properties themselves can be measured in many different ways, e.g., by micromechanical determination of mass (which can be measured, e.g., using mechanical resonance), changes in capacitance, and optical changes. Such sensors must be safe and function in a reliable manner for a long period of time under harsh conditions. Despite numerous efforts, the technical form of such sensors still poses an enormous challenge in terms of the ability of such sensors to resist harmful influences in the implanted state. These sensors can be damaged primarily by unwanted interactions between the sensor surface and the biological medium.

Examples of these interactions include, but are not limited to, the deposition of proteins or the binding or activation of metabolic active cells (blood platelets) on the sensor surface. The adsorption of blood platelets changes, e.g., the chemical environment of the implant by the absorption of O₂ and the release of CO₂, which lowers the measured pH value.

By comparison, sensors implanted in subcutaneous tissue induce cellular attachment and encapsulation by tendinous tissue as the response by the body's self-defense system.

As a result, sensor systems that remain in permanent contact with bodily fluids must be cleaned on a regular basis, which is an elaborate procedure, or they may even need to be replaced entirely or in part, which is likewise an elaborate procedure and renders the sensor information unavailable in the interim.

Furthermore, in general, the measurement principle employed by a chemical sensor, which is based, e.g., on the adsorption of certain molecules or the reaction or degradation products thereof, can stand in the way of long-term use. If additional measures were not taken, such a sensor could be used only to perform a single measurement. To enable the sensor to be used more than once, the adsorbed molecules must be removed.

According to a known solution, a plurality of initially encapsulated sensors is used. A new calibrated sensor is activated, as necessary, when a damaged sensor must be deactivated. The encapsulation of the newly activated sensors is removed only after each of the used sensors is deactivated. This solution is also highly technically complex, of course, and may be uneconomical since a plurality of sensors must be kept on hand and limitations regarding the volumes and surfaces that are available typically come into play with implants. Since implants cannot be made in any size whatsoever, the number of sensors to be kept on hand is greatly limited, thereby also limiting the long-term use or the number of measurements.

A problem to be solved by the present inventive disclosure is therefore that of providing a method and a related implantable system that solves the aforementioned problems.

### SUMMARY

A problem is solved by a method and by an implantable system having the features of the respective independent claim(s). Advantageous developments of the inventive disclosure are the subject matter of the respective dependent claims.

The present disclosure is based on the fundamental idea of providing a method for the reactivation and, optionally, the calibration of an implantable chemical sensor that measures values in bodily fluid in the implanted state, and mainly during on-going operation. It is also based on the idea of cleaning the relevant sensor surface (i.e., the surface with which the sensor interacts with the surrounding fluid in an information-relevant manner) by wetting the sensor surface with a reactivation substance that carries out a chemical reaction on the sensor surface that removes or converts molecules originating in bodily fluid, or the reaction or degradation products thereof, that have sedimented on the sensor, and dewetting the sensor surface of the reactivation substance. The method according to the present disclosure is therefore characterized in that the sensor surface is wetted in situ with a reactivation substance, and is subsequently dewetted thereof.

As an alternative or in addition to the reactivation substance, the sensor can also be brought into contact with a calibration fluid. The sensor can be calibrated using the known chemical and physical properties of the calibration fluid. The calibration fluid is typically tailored to the particular measurement principle employed by the sensor. In the case of a pH sensor, for instance, a buffered solution having a known pH value can be used as the calibration fluid. In a suitable geometric configuration, a plurality of calibration fluids can also be deliberately brought into contact with the sensor, thereby even making it possible to perform multipoint calibration.

The present disclosure makes it possible to perform reversible reactivation and, optionally, calibration of the implanted chemical sensor without removing it from the patient's body. This reactivation enables the sensor to be used for the long term, which is an important prerequisite for use in an implant.

According to one embodiment, the sensor surface can be wetted using electrowetting. This technique makes it possible to change the surface tension of conductive fluids using an electric field. The sensor surface can therefore be wetted with a reactivation substance or a calibration fluid, or both, in a deliberate manner using electrical control.

Furthermore, by employing a suitable geometric configuration of electrodes and energizing electrodes in a suitable temporal manner, electrowetting can be used to move droplets or, more generally, the boundary surface between a fluid electrolyte and a fluid which is not miscible therewith, or a gas. It is therefore possible, e.g., to bring a sensor in contact with an electrolyte, or to remove the electrolyte from the sensor.

As an alternative, other methods, such as, for example, using the action of at least one mechanical actuator, can be used to bring the reactivation substance or a calibration fluid, or both, into contact with the sensor surface and remove it therefrom (or to substantially increase the contact area of the reactivation substance with the sensor surface and to subsequently reduce it in size once more). As such, it is no longer necessary to generate electric fields configured for electrowetting, which is a relatively complex procedure.

A piezo actuator, an electromagnetic actuator, a bimetallic actuator, a hydraulic or pneumatic actuator, a memory effect actuator, or a magnetoresistive actuator can be used as the mechanical actuator in the sensor according to the present disclosure. Of course, other mechanical actuators can be used in the sensor without departing from the spirit and scope of the present invention.

According to an advantageous embodiment of the inventive method, at least one semipermeable outer membrane is installed between the sensor surface and the bodily fluid; and a coupling fluid having direct contact with the sensor surface is disposed there between. The coupling fluid is therefore coupled to the bodily fluid via the outer membrane to protect the sensor against direct action by the bodily fluid. Advantageously, the requirements on the biocompatibility of the sensor or the reactivation substance, or both, are reduced, because there is no direct contact between the sensor and the bodily fluid. Since the reactivation substance or the calibration fluid, or both, is present in the constant volume between the membrane and the sensor surface, the wetting and dewetting of the sensor surface functions with greater accuracy and reliability.

According to a further form of this embodiment, the volume between the outer membrane and the sensor surface can be filled with a plurality of fluids. These fluids can be at least one coupling fluid, at least one reactivation substance, and one or more separating fluids. The fluids are in contact with one another, i.e., they touch one another, but they do not mix since their physical or chemical properties, or both, differ. As a result, it becomes possible to move these fluids using electrowetting. One of the aforementioned mechanical actuators can induce a pressure change in the volume between the outer membrane and the sensor surface to change the concentration of molecules or ions in fluids in the interior using, for example, osmosis and reverse osmosis.

According to another embodiment, a semipermeable inner membrane is also installed between the outer membrane and the sensor surface in the coupling fluid. A pressure change can be induced on the inner membrane by the action of a mechanical actuator. It is therefore possible to change the concentration of molecules or ions within the volume enclosed between the outer membrane and the inner membrane, and between the inner membrane and the sensor surface in a deliberate manner using osmosis and reverse osmosis.

According to another embodiment, another semipermeable membrane that may have other properties is installed between the interior and the outer space. The interior is filled completely with electrolytes (one or more cohesive fluid quantities) and one or more additional non-miscible fluids (e.g., silicone oil). By the skillful application of electrowetting, it is then possible to bring oil or electrolyte into contact with the membranes in a selective manner. A pressure change can be induced in the interior by the action of a mechanical actuator (e.g., piezo). The pressure change can then be used in conjunction with the selective wetting of the membranes with electrolyte or oil to change the concentration of molecules or ions in the electrolyte fluids in a deliberate manner using, for example, osmosis and reverse osmosis.

In another embodiment, a reactivation substance or a calibration fluid, or both, are coupled to the outer or inner, or both, surfaces of the membrane. By way of electrowetting or the action of a mechanical actuator, the outer or the inner, or both, membrane surfaces can be wetted and dewetted to remove molecules originating in the bodily fluid, or the reaction or degradation products thereof, that have sedimented on the membrane surfaces. The sensor as well as the protective membrane can therefore be used for the long term.

If at least one first and one second adjacent outer or inner, or both, membranes are present between the sensor surface and the bodily fluid, the action of at least one membrane can be deactivated by preventing the transfer of molecules or ions through the membrane while wetting the relevant membrane surface, e.g., using electrowetting. Advantageously, the membranes can have different properties, thereby enabling different molecules or ions to pass through the membrane(s). This can be useful, e.g., if the sensor is used to monitor bodily fluid values in different biological environments.

In addition, or as an alternative to regeneration, the sensor surface can be wetted and dewetted using a special fluid having known properties in order to calibrate the sensor.

The present disclosure is also based on the idea of forming an implantable system comprising a sensor, a reactivation substance coupled to the sensor surface, which carries out a suitable cleaning reaction on the sensor surface, and a means for wetting and dewetting the sensor surface with the reactivation substance. In regard to this aspect, the present disclosure makes it possible to provide an implantable system in which a chemical sensor remains operational for long time periods. The reactivation substance enables the sensor surface to be reactivated, e.g., by rinsing or displacing or converting chemical ligands or the combinations thereof on the sensor surface. Furthermore, the properties of the reactivation substance can be defined such that the sensor is calibrated during wetting.

According to one embodiment of the present disclosure, the means for wetting and dewetting comprises a first electrode which is coupled to the reactivation substance, and a second electrode which is coupled to the sensor, wherein the two electrodes together make it possible to wet the sensor surface using electrowetting. Basically, the means for wetting and dewetting therefore comprise a pair of electrodes, wherein the space between the electrodes is filled with a biocompatible fluid that corresponds to the reactivation substance. This fluid can be, for example, silicone oil or another hydrophobic fluid.

According to one variant, the reactivation substance comprises a first and a second fluid having different chemical or physical, or both, properties, wherein the first fluid is moved using electrowetting, and the second fluid (reagent) carries out the chemical reaction on the sensor surface. The first (wetting) fluid can form a capsule (e.g., enclosing a droplet of the second fluid). If the capsule is moved using electrowetting, the fluid contained therein is also moved. Thus, special fluids can be used, which are better suited for the purpose of electrowetting or the chemical reaction with the sensor surface.

As an alternative to electrowetting, the sensor surface can be wetted and dewetted with the reactivation substance using other methods, e.g., a mechanical actuator, e.g., utilizing the piezoelectric effect, wherein the practical advantages of electrical operation likewise apply.

According to one embodiment of the implantable system, the reactivation substance that wets the sensor surface can be a free droplet or a fluid contained in a container, in particular, a gap, a groove or a capillary, possibly coupled to a reservoir of the reactivation substance. The reactivation substance can wet the sensor surface in a deliberate manner via electrowetting or, e.g., the piezoelectric effect. The container is provided with at least one opening in the region of the sensor surface to allow the bodily fluid values to be measured. As an alternative, the opening can be protected by one or more semipermeable membranes, thereby ensuring that the sensor surface is not in direct contact with the bodily fluid. The same effects and advantages of the membranes as those described above result in this case as well, of course.

A few additional device-related aspects result in regard to the aforementioned method-related aspects. For example, the sensor can comprise at least one semipermeable outer membrane between the sensor surface and the bodily fluid, and a coupling fluid that is in direct contact with the sensor surface and is coupled to the bodily fluid via the outer membrane to protect the sensor against direct action by the bodily fluid, wherein the reactivation substance is disposed in the constant volume between the membrane and the sensor surface.

The one or more semipermeable outer membranes can be used as a geometric configuration of a plurality of various outer membrane segments which also have different chemical or physical, or both, properties. By moving the fluids in the volume between the outer membrane segments and the sensor surface, the segments are brought into contact with a type of fluid for which they are permeable or impermeable, thereby selectively activating and deactivating the action of the membrane segments.

It is likewise feasible to provide the sensor with at least one semipermeable inner membrane between the at least one outer membrane and the sensor surface in the coupling fluid, and with a mechanical actuator coupled to the inner membrane to change the concentration of molecules or ions within the volume enclosed between the outer membrane and the inner membrane, and between the inner membrane and the sensor surface using, for example, osmosis and reverse osmosis.

Furthermore, an additional reactivation substance for wetting and dewetting the outer or the inner, or both, membrane surfaces, which is coupled to the membrane surfaces, can be provided by way of electrowetting or the action of a mechanical actuator to remove molecules originating in the bodily fluid, or the reaction or degradation products thereof, that have collected on the membrane surfaces.

Likewise, a reservoir can also be provided for the used reactivation fluid.

Further device-related aspects of the present disclosure result directly from the further method-related aspects described above, and therefore these aspects will not be explained once more.

The sensor used in the above-described method and the implantable system can be any type of chemical sensor which measures values in bodily fluid, such as, for example, a pH sensor, a protein sensor, an electrochemical sensor, an ion concentration sensor, an enzymatic sensor, a micromechanical sensor, an optical sensor, or an interferometric sensor. Sensors are also feasible that operate according to other principles, e.g., to measure glucose, oxygen saturation, CO₂ content, gas content, ions, proteins, antibodies, pH value, hormones, messenger substances, metabolites, pathogens, blood components, clotting factors, cholesterol, nutrients, toxins, or drug components.

Various other objects, aspects and advantages of the present inventive disclosure can be obtained from a study of the specification, the drawings, and the appended claims.

### DESCRIPTION OF THE DRAWINGS

Advantages and useful features of the present disclosure also result from the description that follows of selected exemplary embodiments with reference to the Figures. In the drawings:

FIG. 1 shows a schematic depiction of an implantable system according to the present disclosure;

FIGS. 2A and 2B show schematic depictions of an implantable system comprising a pair of electrodes as means for wetting and dewetting, according to one embodiment of the present disclosure;

FIGS. 3A and 3B show schematic depictions of an implantable system comprising a piezo actuator as means for wetting and dewetting, according to an alternative embodiment of the present disclosure;

FIG. 4 shows a schematic depiction of an implantable system comprising a first and a second fluid, as the reactivation substance, according to another embodiment of the present disclosure;

FIG. 5 shows a schematic depiction of an implantable system comprising a container and a reservoir for the reactivation substance, according to another embodiment of the present disclosure;

FIGS. 6A and 6B show schematic depictions of an implantable system comprising one or more semipermeable membranes between the sensor surface and the bodily fluid, according to another embodiment of the present disclosure;

FIGS. 7A and 7B show a schematic depiction of an implantable system comprising a closed inner volume, a membrane and a coupling fluid, a container and reservoir for the reactivation substance, according to another embodiment of the present disclosure; and

FIG. 8 shows a schematic depiction of an implantable system comprising a closed inner volume, a membrane, and a coupling fluid, a container and reservoir for the reactivation substance, and a container and reservoir for used reactivation substance, according to another embodiment of the present disclosure.

### DETAILED DESCRIPTION OF THE INVENTION

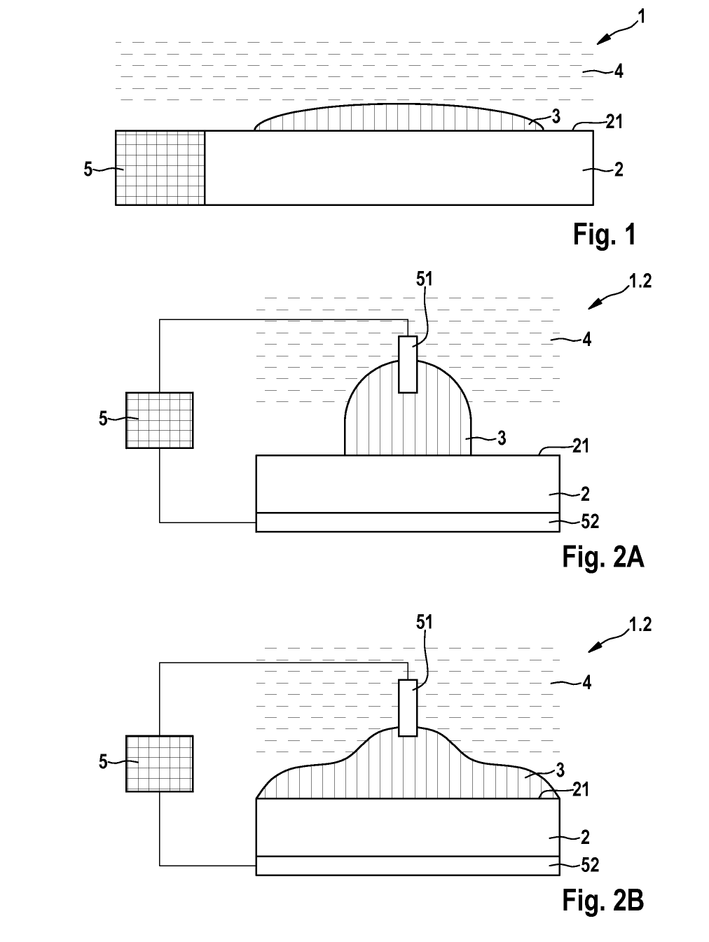

FIG. 1 shows an implantable system 1, comprising a sensor 2 which has a sensor surface 21 which is in contact with bodily fluid 4 of a patient. A reactivation substance 3 that carries out a chemical reaction on the sensor surface 21 is provided on the sensor surface 21. Implantable system 1 also comprises means 5 for wetting and dewetting the sensor surface 21 with the reactivation substance 3 to remove molecules which originated in the bodily fluid 4 and have collected on the sensor 2.

FIGS. 2A and 2B show an implantable system 1.2, in which the sensor surface 21 is wetted and dewetted with the reactivation substance 3 using electrowetting. Means 5 for wetting and dewetting comprises a first electrode 51, which is coupled to reactivation substance 3, and a second electrode 52 which is coupled to the sensor 2. Reactivation substance 3 is a free droplet in this case. The surface tension of reactivation substance 3 is changed by the action of an electric field, thereby transferring it from a first state, in which it covers only a portion of the sensor surface 21 (FIG. 2A), to a second state, in which it wets the entire sensor surface 21 as intended (FIG. 2B).

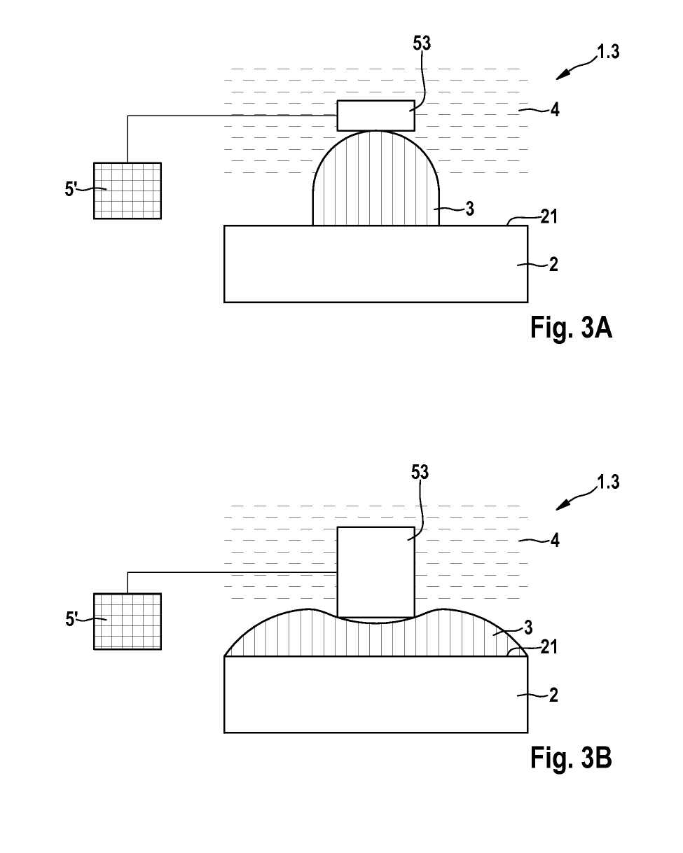

FIGS. 3A and 3B show, as an alternative to the implantable system 1.2 depicted in FIGS. 2A and 2B, another implantable system 1.3, in which the sensor surface 21 is wetted and dewetted with the reactivation substance 3 via the action of the piezoelectric effect. A means 5′ for wetting and dewetting comprises a piezo actuator 53 which is coupled to the reactivation substance 3. Reactivation substance 3 is a free droplet in this case as well. When an electric voltage is applied, the piezo actuator 53 responds with mechanical deformation. As a result, the droplet of reactivation substance 3 deforms such that it wets essentially the entire sensor surface 21 as intended (FIG. 3B).

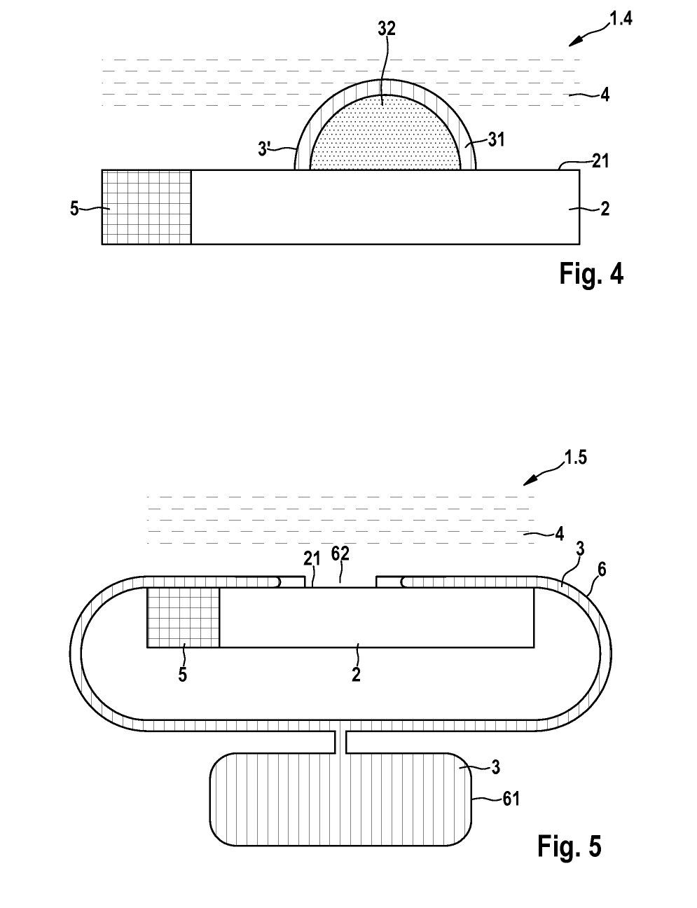

FIG. 4 shows another implantable system 1.4, wherein the reactivation substance 3′ comprises a first and a second fluid 31 and 32. In particular, the first fluid 31 forms a capsule around a droplet of the second fluid 32. While the first fluid 31 is used only for electrowetting, the second fluid 32 is used only for the chemical reaction on the sensor surface 21. If the capsule is moved using electrowetting, the fluid contained therein is also moved and then carries out the chemical reaction on the sensor surface 21.

FIG. 5 shows another implantable system 1.5, wherein the reactivation substance 3 is contained in a capillary 6. A reservoir 61 containing the reactivation substance 3 is coupled to the capillary 6. Capillary 6 is provided with an opening 62 in the region of the sensor surface 21 to connect the bodily fluid 4 to the sensor surface 21.

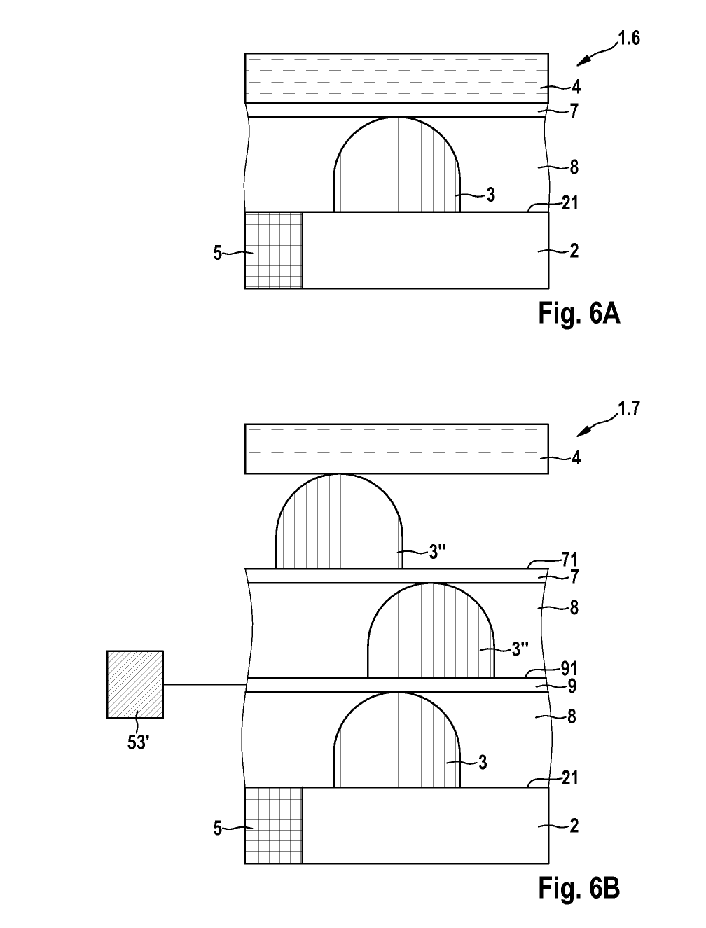

FIG. 6A shows another implantable system 1.6, in which a semipermeable membrane 7 (outer membrane) is disposed between the sensor surface 21 and the bodily fluid 4. A coupling fluid 8 is present, which is in direct contact with the sensor surface 21 and is coupled to the bodily fluid 4 via the membrane 7. As a result, the sensor surface 21 is not in direct contact with the bodily fluid 4, and the reactivation substance 3 is disposed in the constant volume between the membrane 7 and the sensor surface 21.

FIG. 6B shows, as system 1.7, a variant of the implantable system according to FIG. 6A, in which an additional semipermeable membrane 9 (inner membrane) is disposed between the outer membrane 7 and the sensor surface 21 in coupling fluid 8, and wherein a piezo actuator 53′ is coupled to the inner membrane 9 and induces a pressure change on the inner membrane 9. An additional reactivation substance 3″ is coupled to membranes 7 and 9 to wet and dewet the outer and inner membrane surfaces 71 and 91.

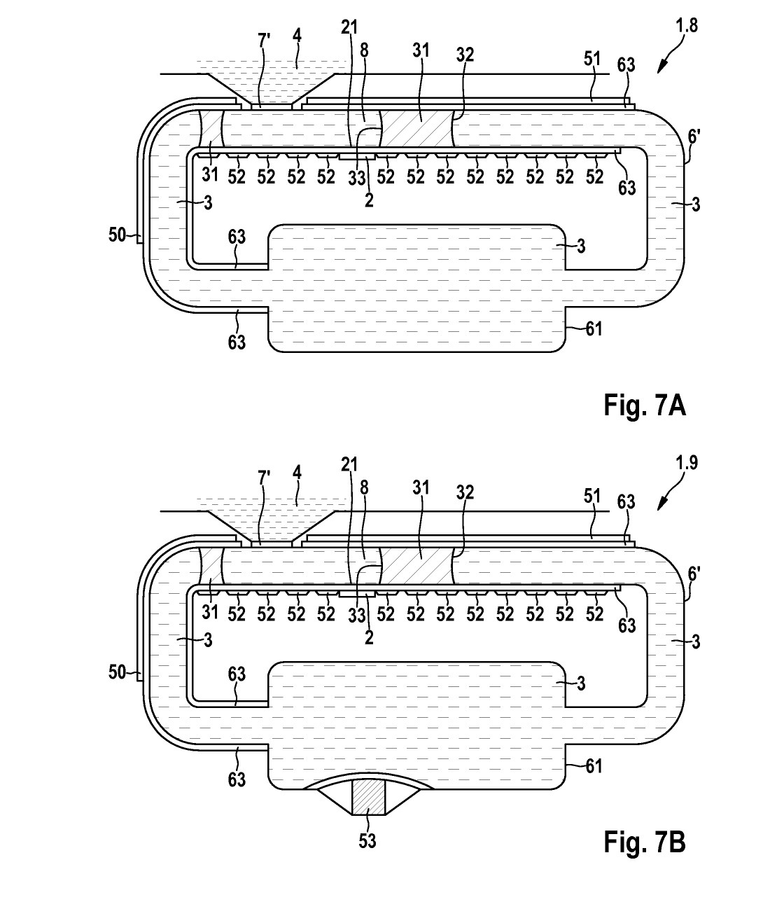

FIG. 7A shows a system 1.8, in which a closed fluid circuit 6 is coupled to a reservoir 61, and the fluid circuit 6 is coupled to the bodily fluid 4 at a point via a semipermeable membrane 7′. Fluid circuit 6 is now filled such that four regions result, which are filled, in alternation, with an electrolyte (3 and 8) and a non-electrically conductive separating fluid 31 (e.g., silicone oil) which is not miscible therewith.

Electrodes 50-52 are now installed along the fluid circuit 6 in a suitable geometric configuration and, optionally, in a repeated sequence. A thin layer 63 has been applied to make the surface of the electrodes 50-52 hydrophobic. By connecting the electrodes 50-52 to a voltage in a deliberate manner, the wetting of surface 63 directly above the electrodes at boundary surface 32 between electrolyte and non-electrolyte can be varied such that the boundary surface 32 moves in a direction in a deliberate manner. Along therewith, the entire fluid column 3, 31, 8, 31 then moves within the closed circuit 6.

Depending on the position of the boundary surface 32, configurations A-D described below can be attained: A) Sensor 2 is coupled to coupling fluid 8 via semipermeable membrane 7 and bodily fluid 4. Separating fluid 31 separates the coupling fluid 8 from the fluid 3. B) Sensor 2 is enclosed entirely by fluid 31, thereby enabling the sensor 2 to be cleaned or calibrated if necessary. Depending on the geometric configuration of the semipermeable membrane 7, it can be in contact with the coupling fluid 8 or the separating fluid 31. C) Sensor 2 is enclosed entirely by fluid 3 which can have a cleaning or regenerative effect. Membrane 7 is surrounded by separating fluid 31, thereby preventing an exchange of substances in the fluid 3 with the bodily fluid 4. D) Sensor 2 and membrane 7 are in contact with fluid 3, thereby possibly resulting in a cleaning or regenerative effect.

FIG. 7B shows system 1.9 as a modification of system 1.8, in which a mechanical actuator 53 that generates pressure is installed within the closed inner volume, and is used in combination with the semipermeable membrane 7 to induce osmosis and reverse osmosis.

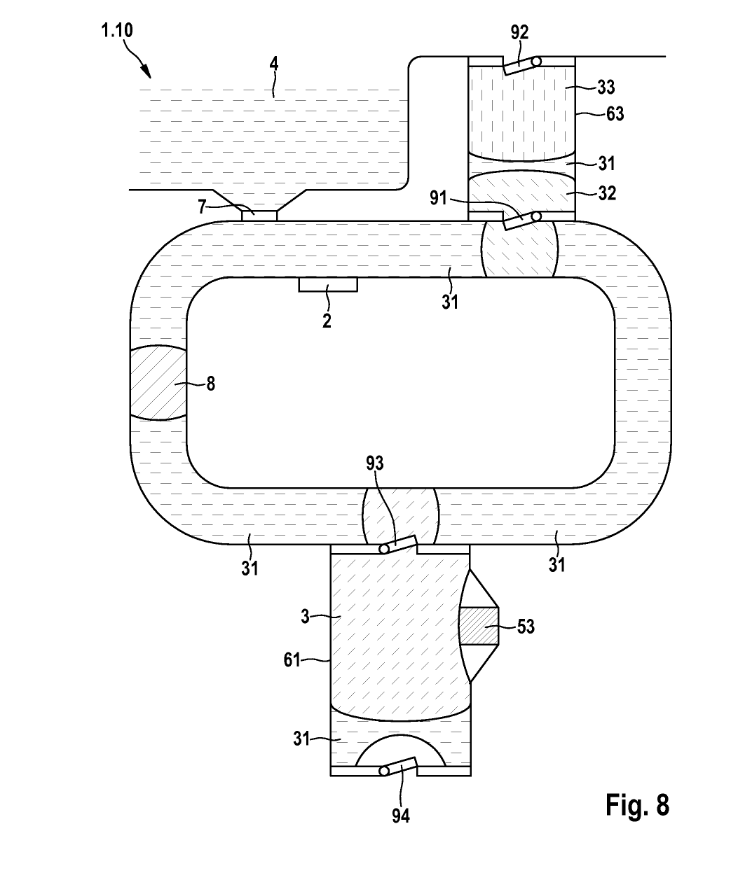

FIG. 8 shows system 1.10 as a modification of system 1.9, in which a reservoir 63 for used reactivation fluid 32, which is closed by a valve 91, is contained within the closed inner volume. A biocompatible, non-miscible fluid 31 (e.g., silicone oil) or an additional valve 92 are used as the pressure compensation element. To receive used reactivation fluid, a biocompatible fluid (e.g., saline solution) can be released into the surroundings through the valve 92. In the embodiment shown, reservoir 61 also comprises a valve 94 and a separating fluid 31 for equalizing pressure with the outer region. Valve 93 regulates the release of regeneration fluid 3 into the interior. By arranging the electrodes (which are omitted from this illustration to ensure clarity) in a suitable configuration, the entire fluid column comprised of fluids 3, 31, 32 and 8 can be moved using electrowetting, and the various fluids can be brought into the position at the valves or the sensor 2 so that fluids can then be moved through the particular valves using the mechanical actuator 53.

The exemplary embodiment of the present disclosure is not limited to the above-described examples and emphasized aspects but, rather, may appear in a large number of modifications that lie within the scope of handling by a person skilled in the art.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/We claim:

1. A method for the operation of an implantable chemical sensor that measures values in bodily fluid, comprising the following steps:
wetting a sensor surface with a reactivation substance that carries out a chemical or physical reaction on the sensor surface that removes or converts molecules originating in the bodily fluid, or the reaction or degradation products thereof, that have sedimented on the sensor; and
dewetting the sensor surface of the reactivation substance,
wherein the sensor surface is wetted and dewetted with the reactivation substance in situ.

2. The method according to claim 1, wherein electrowetting is used for wetting the sensor surface.

3. The method according to claim 1, wherein wetting the sensor surface is carried out by an action of a mechanical actuator.

4. The method according to claim 1, further comprising the steps of:
installing a semipermeable outer membrane between the sensor surface and the bodily fluid, and a coupling fluid that is in direct contact with the sensor surface, wherein the coupling fluid is coupled to the bodily fluid via the outer membrane to protect the sensor against direct action by the bodily fluid, wherein wetting or dewetting the sensor surface, the outer membrane, or both, is carried out by using the reactivation substance contained in the constant volume between the outer membrane and the sensor surface.

5. The method according to claim 4, further comprising the steps of:
filling the volume between the outer membrane and the sensor surface with a plurality of fluids, including a coupling fluid, a reactivation substance, and optional separating fluids, which touch one another but do not mix because of different physical, chemical, or both, properties, wherein moving the plurality of fluids is carried out by using electrowetting.

6. The method according to claim 4, further comprising the steps of:
inducing a pressure change in the volume between the outer membrane and the sensor surface using a mechanical actuator to change the concentration of molecules or ions in fluids in the interior using osmosis and reverse osmosis.

7. The method according to claim 4, further comprising the steps of:
installing a semipermeable inner membrane between the outer membrane and the sensor surface in the coupling fluid, and inducing a pressure change on the inner membrane using a mechanical actuator to change the concentration of molecules or ions within the volume enclosed between the outer membrane and the inner membrane, and between the inner membrane and the sensor surface, using osmosis and reverse osmosis.

8. The method according to claim 7, further comprising the steps of:
wetting and dewetting the outer, the inner, or both, membrane surfaces with an additional reactivation substance coupled to the membrane surfaces using electrowetting or the action of a mechanical actuator to remove molecules originating in the bodily fluid, or the reaction or degradation products thereof, that have sedimented on the membrane surfaces.

9. An implantable system comprising:
a sensor that measures values in bodily fluid;
a reactivation substance that is coupled to the sensor surface and carries out a chemical reaction on the sensor surface that removes molecules originating in the bodily fluid, or the reaction or degradation products thereof, that have sedimented on the sensor; and
a means for wetting and dewetting the sensor surface with the reactivation substance.

10. The system according to claim 9, wherein the means for wetting and dewetting comprises a first electrode, which is coupled to the reactivation substance, and a second electrode for electrowetting, which is coupled to the sensor.

11. The system according to claim 10, wherein the reactivation substance is a biocompatible fluid.

12. The system according to claim 11, wherein the biocompatible fluid is silicone oil.

13. The system according to claim 11, wherein the reactivation substance comprises a first and a second fluid having different chemical or physical properties, or combination thereof, wherein the first fluid is moved using electrowetting, and the second fluid carries out the chemical reaction on the sensor surface, and wherein the first fluid encloses the second fluid, and therefore the second fluid is moved along by the motion of the first fluid.

14. The system according to claim 9, wherein the means for wetting and dewetting comprises a mechanical actuator which is coupled to the reactivation substance.

15. The system according to claim 9, wherein the reactivation substance is present as a free droplet.

16. The system according to claim 9, further comprising a container, a gap, a groove, or a capillary that contains the reactivation substance and is coupled to a reservoir of the reactivation substance.

17. The system according to claim 9, further comprising a semipermeable outer membrane between the sensor surface and the bodily fluid, and a coupling fluid that is in direct contact with the sensor surface and is coupled to the bodily fluid via the outer membrane to protect the sensor against direct action by the bodily fluid, wherein the reactivation substance is disposed in a constant volume between the outer membrane and the sensor surface.

18. The system according to claim 17, further comprising a semipermeable inner membrane between the outer membrane and the sensor surface in the coupling fluid, and further comprising a mechanical actuator coupled to the inner membrane to change the concentration of molecules or ions within the volume enclosed between the outer membrane and the inner membrane, and the volume enclosed between the inner membrane and the sensor surface, using osmosis and reverse osmosis.

19. The system according to claim 18, further comprising an additional reactivation substance, which is coupled to the membrane surfaces, for wetting and dewetting the outer or the inner, or both, membranes surfaces using electrowetting or the action of a mechanical actuator to remove molecules originating in the bodily fluid, or the reaction or degradation products thereof, that have sedimented on the membrane surfaces.

20. The system according to claim 9, wherein a reservoir for the used reactivation fluid is also provided.
